(19) United States
(12) Patent Application Publication
(10) Pub. No.: US 2021/0307640 A1
(43) Pub. Date: Oct. 7, 2021

(54) DEVICE FOR IDENTIFYING A POSITION OF A CATHETER

(71) Applicant: EPMAP System GmbH, Herdecke (DE)

(72) Inventors: Jörg Ströbel, Nürnberg (DE); Manfred Piechura, Herdecke (DE)

(21) Appl. No.: 17/267,132

(22) PCT Filed: Mar. 11, 2020
(86) PCT No.: PCT/IB2020/052086

§ 371 Date: Feb. 9, 2021

(30) Foreign Application Priority Data
Mar. 12, 2019 (GB) 1903330.7

(51) Int. Cl. A61B 5/06; A61B 5/00

(52) U.S. Cl. CPC A61B 5/063; A61B 5/6801; A61B 5/6846

(57) ABSTRACT

The application provides a catheter positioning device for a catheter with a detection electrode. The device includes three pairs of excitation electrodes. The excitation electrode pairs are respectively lie on an X-axis, a Y-axis, and a Z-axis of a coordinate system. The device also includes a signal generator for providing excitation signals to the respective pairs of excitation electrodes. The device further includes a computing processing unit for measuring differential voltage indicative of impedance between the detection electrode and a first pair of excitation electrodes for determining an X coordinate of a position of the catheter, for measuring differential voltage indicative of impedance between the detection electrode and a second pair of excitation electrodes for determining a Y coordinate of the position, and for measuring differential voltage indicative of impedance between the detection electrode and a third pair of excitation electrodes for determining a Z coordinate of the position.

The application relates to catheters. In particular, the application relates to positioning of one or more catheters within a body of a patient.

Catheters, which are used for inserting into a vessel of a patient to carry electrical signals to and from the patient, are used in various applications. For example, cardiac catheters are inserted within a blood vessel into a patient's heart to detect cardiac electrical signals, to apply electrical stimulation for diagnostic testing, and to apply treatment signals, such as tissue ablation signals, for eliminating the source of an arrhythmia.

It is an object of the application to provide an improved device and method for identifying a position of a catheter within a body of a patient.

The application provides an improved device for identifying a position of a catheter within a body of a patient. The catheter refers to a medical device that can be inserted in the body of the patient to treat diseases or perform a surgical procedure. The catheter is often a thin flexible tube with one or more electrodes provided at a distal end of the flexible tube. The electrodes include a detection electrode. The thin tube can be inserted into a body cavity, duct, or vessel to reach a disease area that can be treated by ablation energy delivered from the electrodes. In order to reach the disease area, a doctor uses the improved device to identify the current position of the catheter and move the catheter accordingly to the disease area.

The device includes a first pair of excitation electrodes that lie on an X-axis of a coordinate system and a second pair of excitation electrodes that lie on a Y-axis of the coordinate system. The device further includes a third pair of excitation electrodes that lie on a Z-axis of the coordinate system. The coordinate system uses the three axes or coordinates X, Y, Z to uniquely determine a position of a point or an electrode of the catheter in a three-dimensional space within the body. The three axes X, Y, Z are not necessary to be mutually orthogonal. They often intersect at an origin. This means that the first pair of excitation electrodes, the second pair of excitation electrodes, and the third pair of excitation electrodes are provided on predetermined positions such that the three axes X, Y, Z can intersect at an origin.

The device also includes a signal generator, which is electrically connected to the first pair of excitation electrodes, to the second pair of excitation electrodes, to the third pair of excitation electrodes. The signal generator is adapted to generate excitation signals, such as sine waves. In other word, the signal generator is configured to provide a first excitation signal to the first pair of excitation electrodes, to provide a second excitation signal to the second pair of excitation electrodes, and to provide a third excitation signal to the third pair of excitation electrodes. The excitation signal is also called localization signal.

The device further includes a computing processing unit. The computing processing unit is adapted to measure differential voltage between the detection electrode and the first pair of excitation electrodes. The differential voltage indicates an impedance between the detection electrode and the first electrode pair. The differential voltage indicative of impedance is used to determine an X coordinate of the position of the detection electrode of the catheter. The differential voltage is a periodic voltage waveform that corresponds to the first excitation signal. The periodic voltage waveform can be mathematically represented by a phase and a magnitude. If the phase is in phase with the first excitation signal provided to the first pair of excitation electrodes, it indicates that the detection electrode is positioned closer to one of the excitation electrodes. If the phase is out of phase with the first excitation signal, it indicates that the detection electrode is positioned closer to the other one of the excitation electrodes. The magnitude indicates the relative proximity of the detection electrode to each of the excitation electrodes. If the magnitude is zero, it indicates that the detection electrode is equidistantly positioned between the excitation electrodes.

The computing processing unit is also adapted to measure differential voltage indicative of impedance between the detection electrode and the second pair of excitation electrodes for determining a Y coordinate of the position of the detection electrode of the catheter. It is also adapted to measure differential voltage indicative of impedance between the detection electrode and the third pair of excitation electrodes for determining a Z coordinate of the position of the detection electrode of the catheter.

The first pair of excitation electrodes, the second pair of excitation electrodes, and the third pair of excitation electrodes can be patch electrodes, which are adapted for external attachment to the body of the patient. Such patch electrodes are easy and convenient to use.

The excitation signals can have different frequencies for different excitation electrode pairs. This means that the first excitation signal has a first frequency, the second excitation signal has a second frequency, and the third excitation signal has a third frequency. Different frequencies can minimize any cross-axis interference.

The coordinate system can have an origin that is located at a body cavity, which can be a heart chamber.

The application also provides a method for identifying a position of a catheter within a body of a patient. The method includes a step of positioning a detection electrode on the catheter. The method also includes a step of positioning a first pair of excitation electrodes such that the first pair of excitation electrodes lie on an X-axis of a coordinate system, followed by a step of positioning a second pair of excitation electrodes such that the second pair of excitation electrodes lie on a Y-axis of the coordinate system, and followed by a step of positioning a third pair of excitation electrodes such that the third pair of excitation electrodes lie on a Z-axis of the coordinate system. In subsequent steps, a first excitation signal is provided to the first pair of excitation electrodes, a second excitation signal is provided to the second pair of excitation electrodes, and a third excitation signal is provided to the third pair of excitation electrodes. The method further includes a step of measuring differential voltage indicative of impedance between the detection electrode and the first pair of excitation electrodes for determining a X coordinate of the position of the detection electrode of the catheter, followed by a step of measuring differential voltage indicative of impedance between the detection electrode and the second pair of excitation electrodes for determining a Y coordinate of the position of the detection electrode of the catheter, and followed by a step of measuring differential voltage indicative of impedance between the detection electrode and the third pair of excitation electrodes for determining a Z coordinate of the position of the detection electrode of the catheter.

The method can further include providing the first pair of excitation electrodes, the second pair of excitation electrodes, and the third pair of excitation electrodes as patch electrodes, which are adapted for external attachment to the body of the patient.

The provision of excitation signals can include providing the first excitation signal with a first frequency, providing the second excitation signal with a second frequency, and providing the third excitation signal with a third frequency.

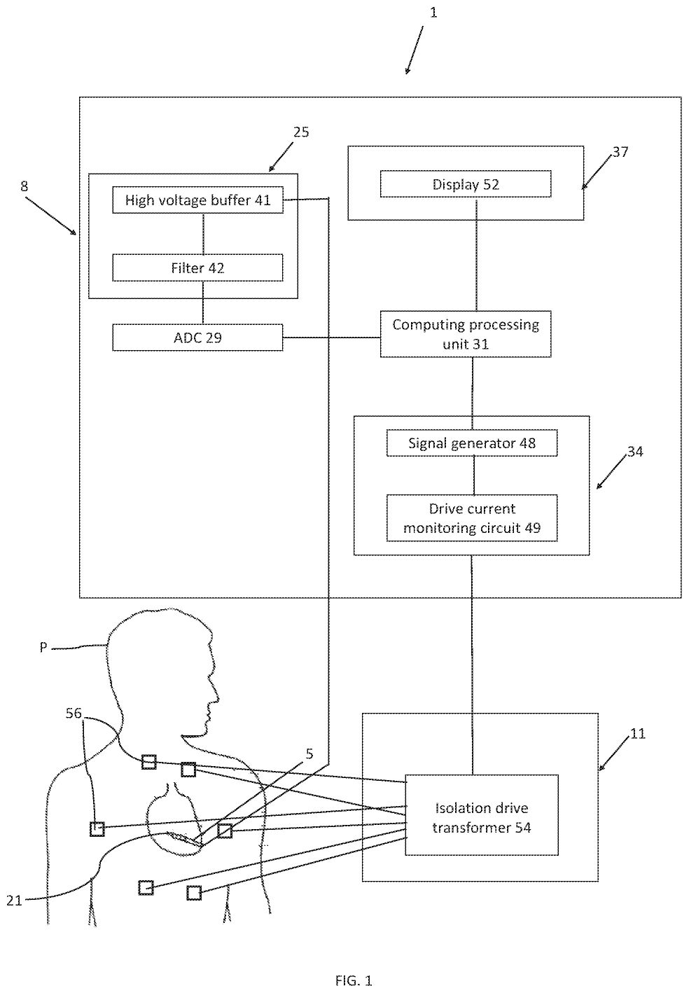

FIG. 1 illustrates a block diagram of a catheter positioning system,

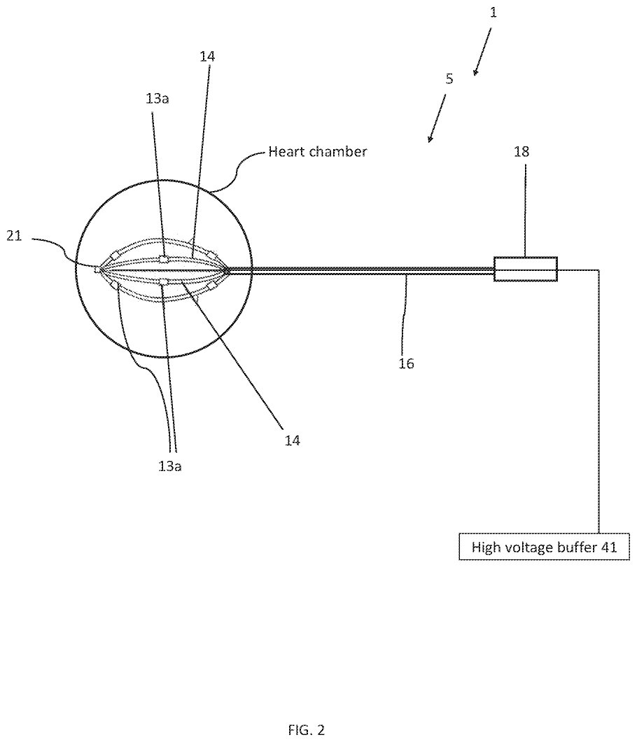

FIG. 2 illustrates a catheter of the catheter positioning system of FIG. 1,

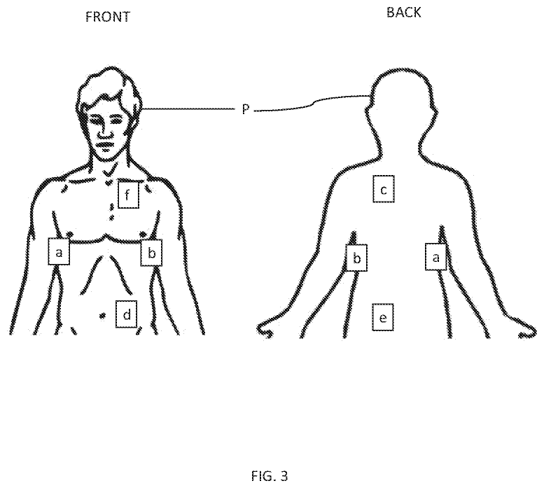

FIG. 3 illustrates a drawing of placement of patch electrodes of the catheter positioning system of FIG. 1 on a patient, and

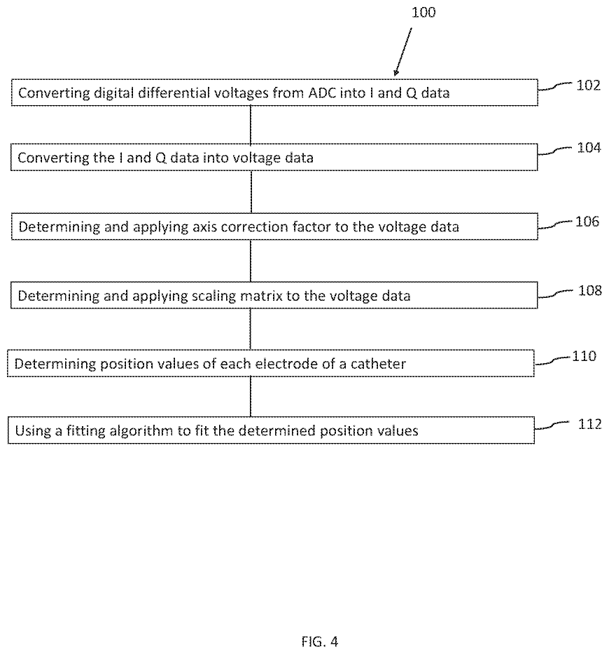

FIG. 4 illustrates a flow chart of a method for determining positions of electrodes of the catheter of FIG. 2 within a body of the patient.

In the following description, details are provided to describe embodiments of the application. It shall be apparent to one skilled in the art, however, that the embodiments may be practiced without such details.

Some parts of the embodiments have similar parts. The similar parts may have same names or similar part numbers. The description of one part applies by reference to another similar part, where appropriate, thereby reducing repetition of text without limiting the disclosure.

FIG. 1 shows a catheter positioning system 1 for guiding a catheter to move to a predetermined position.

The catheter positioning system 1 includes a catheter 5, an information console 8, and a patient interface module 11. The catheter 5 is electrically and/or optically connected to the information console 8, which is electrically connected to the patient interface module 11.

As better seen in FIG. 2, the catheter 5 includes an electrode array 13, an elongate flexible shaft 16, and a handle 18. The electrode array 13 is attached to a distal end of the shaft 16, which is connected to the handle 18. The electrode array 13 is electrically and/or optically connected to the information console 8, as shown in FIG. 1.

The electrode array 13 includes a plurality of electrode supporting members 14 and a plurality of biopotential electrodes 13a, which are provided on the electrode supporting members 14. The biopotential electrodes 13a are arranged in a basket array. The electrode array 13 also include a detection electrode 21, which is located at a tip of the catheter 5. The detection electrode 21 is electrically connected to the information console 8.

The information console 8 includes a signal filtering module 25, an analog-to-digital converter (ADC) module 29, a computing processing unit 31, a signal generation module 34, and a user interface (UI) module 37. The signal filtering module 25 is electrically connected to the catheter 5 and to the ADC module 29, which is electrically connected to the computing processing unit 31. The computing processing unit 31 is electrically connected to the signal generation module 34 and to the UI module 37. The signal generation module 34 is electrically connected to the patient interface module 11.

The signal filtering module 25 includes a high voltage buffer 41 and a high frequency bandpass filter 42. The high voltage buffer 41 is electrically connected to the catheter 5 and to the filter 42. The filter 42 is electrically connected to the ADC module 29. The high voltage buffer 41 has, for example, +−100 volt rails. The filter 42 has a passband frequency range of about 10 kilo hertz (kHz) to 100 kHz. Preferably, the filter 42 has low noise with a gain of, for example, one.

The ADC module 29 includes a plurality of ADCs. Each ADC is configured to have high sampling rate of, for example, about 600 kHz.

The computing processing unit 31 includes one or more microprocessors, and one or more memory modules.

The signal generation module 34 includes a signal generator 48 and a drive current monitor circuit 49. The signal generator 48 is electrically connected to the computing processing unit 31 and to the drive current monitor circuit 49. The drive current monitor circuit 49 is electrically connected to the patient interface module 11.

The signal generator 48 is a direct digital synthesizer, which is configured to generate periodic signals, such as sine waves, with different frequencies between, for example, 20 kilo hertz (kHz) and 80 kHz. In one embodiment, the signal generator 48 is configured to generate periodic signals with three different frequencies of about 36 kHz, 45 kHz, and 51 kHz.

The drive current monitor circuit 49 is configured to monitor and to maintain an electric current provided to the patient interface module 11.

The UI module 37 includes a display 52 with one or more user interface mechanisms, such as a touchscreen, mouse, keyboard, light pen, track ball, microphone.

The patient interface module 11 includes a patient isolation drive transformer 54 and a set of patch electrodes 56. The patient isolation drive transformer 54 is electrically connected to the drive current monitor circuit 49 and to the patch electrodes 56. The patch electrodes 56 are placed on a body of a patient P.

The patient isolation drive transformer 54 is configured to isolate localization signals from other parts of the catheter positioning system 1.

The set of patch electrodes 56 include a first pair of patch electrodes 56X1, 56X2, a second pair of patch electrodes 56Y1, 56Y2 and a third pair of patch electrodes 56Z1, 56Z2. The patch electrodes 56 are also called localization electrodes.

In use, the three pairs of patch electrodes 56 are placed at predetermined positions of a body of a patient P. FIG. 3 shows a front view and a back view of the patient P with the patch electrodes 56 being placed on the body.

The first pair of the patch electrodes 56X1, 56X2 are placed on ribs of the patient P at locations a and b of FIG. 3, wherein the locations a and b are separated by a predetermined distance. The patch electrodes 56X1, 56X2 provide a X axis within the body, wherein the patch electrodes 56X1, 56X2 lie on two ends of the X-axis. The second pair of the electrodes 56Y1, 56Y2 are respectively placed on an upper back and a lower abdomen of the patient P at locations c and d of FIG. 3, wherein the locations c and d are separated by substantially the same predetermined distance. The electrodes 56Y1, 56Y2 provide a Y axis within the body, wherein the patch electrodes 56Y1, 56Y2 lie on two ends of the Y-axis. The third pair of the electrodes 56Z1, 56Z2 are respectively placed on a lower back and an upper chest of the patient P at locations e and f of FIG. 3, wherein the locations e and f are separated by substantially the same predetermined distance. The electrodes 56Z1, 56Z2 provide a Z axis within the body, wherein the electrodes 56Z1, 56Z2 lie on two ends of the Z-axis.

In short, the three axes X, Y, Z have a similar length. The patch electrodes 56 define a coordinate system with three axes X, Y, Z, wherein each pair of patch electrodes 56 defines one axis. The three axes X, Y, Z intersect at a position or an origin, which corresponds to a heart chamber of the patient P.

The signal generator 48 is intended for generating multiple waveforms with different frequencies for each pair of the patch electrodes 56. In one implementation, the signal generator 48 generates a first localization signal with a frequency of about 36 kHz for the first pair of patch electrodes 56X1, 56X2, a second localization signal with a frequency of about 45 kHz for the second pair of patch electrodes 56Y1, 56Y2, and a third localization signal with a frequency of about 51 kHz for the third pair of patch electrodes 56Z1, 56Z2. The localization signals are then transmitted to the drive current monitoring circuit 49.

The drive current monitoring circuit 49 later receives the localization signals. It acts to provide a feedback system for monitoring and maintaining a predetermined electric current of the localization signals. The localization signals afterward travel to the patient isolation drive transformer 54 of the patient interface module 11.

The patient isolation drive transformer 54 acts to isolate the localization signals from other parts of the catheter positioning system 1 to prevent current leakage, which can result in degrading signals provided to the patch electrodes 56. The patient isolation drive transformer 54 also acts to maintain a high isolation between the three localization signals. Furthermore, the isolation drive system 54 also serves to maintains simultaneous output of the localization signals on all the patch electrode pairs 56X1, 56X2, 56Y1, 56Y2, 56Z1, and 56Z2.

The catheter 5 is later inserted into a body of a patient and advanced through a body vessel, such as a femoral vein or other blood vessel towards a body space, such as a chamber of the heart.

The detection electrode 21 afterward receives the localization signals from the patch electrode pairs 56X1, 56X2, 56Y1, 56Y2, 56Z1, and 56Z2. The received localization signals are later transmitted to the high voltage buffer 41.

The high voltage buffer 41 then allows the localization signals to pass through it to reach the filter 42.

The filter 42 later allows the localization signals to pass through it to reach the ADC module 29.

The ADC module 29 afterward converts the filtered localization signals into digital information and then transmits the converted digital information to the computing processing unit 31.

The computing processing unit 31 later receives the digital information about the localization signals. The computing processing unit 31 then execute instructions according to a signal processing algorithm to process the received digital information. The processing results are later displayed on the display 52 graphically in 2D, 3D, or a combination of 2D and 3D.

FIG. 4 shows a flow chart 100 of the signal processing algorithm.

The signal processing algorithm include a step 102 of an IQ demodulator software module analysing magnitudes and phases of the received digital information to generate I and Q data of differential voltages between the patch electrodes 56X1, 56X2, 56Y1, 56Y2, 56Z1, and 56Z2 and the detection electrode 21.

The I and Q data is then converted into voltage data, which corresponds to differential voltages between the patch electrodes 56X1, 56X2, 56Y1, 56Y2, 56Z1, and 56Z2 and the detection electrode 21, in a step 104.

The differential voltage between the electrode 56X1 and the detection electrode 21 indicates an impedance between the detection electrode 21 and the electrode pairs 56X1. The differential voltage between the electrode 56X2 and the detection electrode 21 indicates another impedance between the detection electrode 21 and the electrode pairs 56X2. The differential voltage indicative of impedance is used to determine a X coordinate of the position of the detection electrode 21, which is relative to the X-axis. If the differential voltage has a phase that is in phase with the localization signal that is applied to the electrodes 56X1, 56X2, it indicates that the detection electrode 21 is positioned closer to one of the electrodes 56X1, 56X2. If the phase of the differential voltage is out of phase with the localization signal that is applied to the electrodes 56X1, 56X2, it indicates that the detection electrode 21 is positioned closer to the other one of the electrodes 56X1, 56X2. If the magnitude of the differential voltage is zero, it indicates that the detection electrode 21 is equidistantly positioned between the electrodes 56X1, 56X2. The magnitude indicates the relative proximity of the detection electrode 21 to each of the electrodes 56X1, 56X2.

Similarly, the differential voltage indicative of impedance between the electrode pairs 56Y1, 56Y2 and the detection electrode 21 is used to determine a Y coordinate of the position of the detection electrode 21, which is relative to the Y-axis.

The differential voltage indicative of impedance between the electrode pairs 56Z1, 56Z2 and the detection electrode 21 is used to determine a Z coordinate of the position of the detection electrode 21, which is relative to the Z-axis.

In a subsequent step 106, an axis correction factor is determined based on a known shape of the electrode array 13 and applied to the voltage data. For example, if the basket shape of the electrode array 13 is incorrect, one or more axes X, Y, Z of the patch electrodes 56 are rotated, scaled, and/or deskewed until a proper basket shape is achieved by a user manipulating a corresponding image on the display 52 using the user mechanisms of the user interface module 37.

A scaling matrix is later determined based on the known shape of the electrode array 13 and applied to the voltage data. If a length or size of the electrode array 13 is not right, based on the known proportions of the electrode array 13, one or more of the axes X, Y, Z are scaled accordingly until a proper corresponding size is achieved, in a step 108.

In a next step 110, position values of the electrodes of the electrode array 13 are determined, and they are checked such that the corresponding voltage values are corrected according to steps 106 and 108.

A fitting algorithm is afterward performed to fit the calculated electrode positions to the known basket array configuration of the electrode array 13, in a step 112.

Based on the displayed positions of the electrode array 13 of the catheter 5 on the display 52, a user later advances the catheter 5 through the body vessel until it reaches the chamber of the heart.

The biopotential electrodes 13a of the catheter 5 then collect biopotential signals and deliver RF ablation energy for treatment.

Although the above description contains much specificity, this should not be construed as limiting the scope of the embodiments but merely providing illustration of the foreseeable embodiments. The above stated advantages of the embodiments should not be construed especially as limiting the scope of the embodiments but merely to explain possible achievements if the described embodiments are put into practice. Thus, the scope of the embodiments should be determined by the claims and their equivalents, rather than by the examples given.

### REFERENCE LIST

1 catheter positioning system
5 catheter
8 information console
11 patient interface module
13 electrode array
13a biopotential electrode
14 electrode supporting members
16 shaft
18 handle
21 detection electrode
25 signal filtering module
29 analog-to-digital converter module
31 computing processing unit
34 signal generation module
37 user interface module
41 buffer
42 filter
48 signal generator
49 drive current monitor circuit
52 display
54 patient isolation drive transformer
56 patch electrodes
56X1 patch electrode
56X2 patch electrode
56Y1 patch electrode
56Y2 patch electrode
56Z1 patch electrode
56Z2 patch electrode
100 flow chart
102 step
104 step
106 step
108 step
110 step
112 step
a location
b location
c location
d location
e location
f location
P patient

1. A device for identifying a position of a catheter within a body of a patient, wherein the catheter comprises a detection electrode, the device comprising:
a first pair of excitation electrodes lying on an X-axis of a coordinate system;
a second pair of excitation electrodes lying on a Y-axis of the coordinate system;
a third pair of excitation electrodes lying on a Z-axis of the coordinate system;
a signal generator for providing a first excitation signal to the first pair of excitation electrodes, a second excitation signal to the second pair of excitation electrodes, and a third excitation signal to the third pair of excitation electrodes; and
a computing processing unit for measuring differential voltage indicative of impedance between the detection electrode and the first pair of excitation electrodes for determining an X coordinate of the position of the catheter, for measuring differential voltage indicative of impedance between the detection electrode and the second pair of excitation electrodes for determining a Y coordinate of the position of the catheter, and for measuring differential voltage indicative of impedance between the detection electrode and the third pair of excitation electrodes for determining a Z coordinate of the position of the catheter.

2. The device according to claim 1, wherein the first pair of excitation electrodes, the second pair of excitation electrodes, and the third pair of excitation electrodes are patch electrodes, which are adapted for external attachment to the body of the patient.

3. The device according to claim 1, wherein the first excitation signal has a first frequency, the second excitation signal has a second frequency, and the third excitation signal has a third frequency.

4. The device according to claim 1, wherein an origin of the coordinate system is located at a body cavity.

5. The device according to claim 4, wherein the body cavity is a heart chamber.

6. A method for identifying a position of a catheter within a body of a patient, the method comprising:
positioning a detection electrode on the catheter;
positioning a first pair of excitation electrodes such that the first pair of excitation electrodes lie on an X-axis of a coordinate system;
positioning a second pair of excitation electrodes such that the second pair of excitation electrodes lie on a Y-axis of the coordinate system;
positioning a third pair of excitation electrodes such that the third pair of excitation electrodes lie on a Z-axis of the coordinate system;
providing a first excitation signal to the first pair of excitation electrodes;
providing a second excitation signal to the second pair of excitation electrodes;
providing a third excitation signal to the third pair of excitation electrodes;
measuring differential voltage indicative of impedance between the detection electrode and the first pair of excitation electrodes for determining a X coordinate of the position of the catheter;
measuring differential voltage indicative of impedance between the detection electrode and the second pair of excitation electrodes for determining a Y coordinate of the position of the catheter; and
measuring differential voltage indicative of impedance between the detection electrode and the third pair of excitation electrodes for determining a Z coordinate of the position of the catheter.

7. The method according to claim 6, further comprising providing the first pair of excitation electrodes, the second pair of excitation electrodes, and the third pair of excitation electrodes as patch electrodes, which are adapted for external attachment to the body of the patient.

8. The method according to claim 6, wherein the provision of first excitation signal comprises providing the first excitation signal with a first frequency, the provision of second excitation signal comprises providing the second excitation signal with a second frequency, and the provision of third excitation signal comprises providing the third excitation signal with a third frequency.
